(19) United States
(12) Patent Application Publication
(10) Pub. No.: US 2017/0022179 A1
(43) Pub. Date: Jan. 26, 2017

(54) COMPOSITIONS, DOSAGES, AND METHODS OF USING TETRAHYDROCANNABINOL DERIVATIVES

(71) Applicant: Corbus Pharmaceuticals, Inc., Newton, MA (US)

(72) Inventors: Mark Tepper, Newton, MA (US); Kollol PAL, Needham, MA (US)

(21) Appl. No.: 15/284,106

(22) Filed: Oct. 3, 2016

Related U.S. Application Data
(63) Continuation of application No. 13/878,007, filed on Aug. 19, 2013.
Application No. 13/878,007 is a continuation of application No. PCT/US11/54985, filed on Oct. 5, 2011.
(60) Provisional application No. 61/390,089, filed on Oct. 5, 2010.
(60) Provisional application No. 61/390,093, filed on Oct. 5, 2010.

(51) Int. Cl. C07D 311/80

(52) U.S. Cl. CPC C07D 311/80

(57) ABSTRACT

Dosage forms of compounds of formula I, e.g., ajulemic acid, and methods of delivering these compounds and using these compounds to treat or prevent a CB1/CB2 associated disease are disclosed.

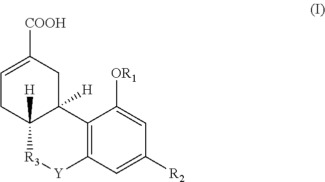

### CROSS-REFERENCE TO RELATED APPLICATIONS

This application claims priority to U.S. Provisional Application Nos. 61/390,089 and 61/390,093, both filed Oct. 5, 2010, each of which is hereby incorporated by reference.

### BACKGROUND OF THE INVENTION

Tetrahydrocannabinol (THC) is the major psychoactive constituent of marijuana. In addition to mood-altering effects, THC has been reported to exhibit other activities, some of which may have therapeutic value including analgesia and anti-emesis. The potential therapeutic value of THC has led to a search for related compounds which minimize the psychoactive effects, while retaining the activities of potential medicinal value.

For example, (6aR,10aR)-3-(1,1-dimethylheptyl)-Δ8-tetrahydro-cannabinol-9-carboxylic acid, also known as ajulemic acid, either alone or in combination with other agents, is a candidate for the treatment of diseases such as osteoarthritis, systemic lupus erythematosus, post herpetic neuralgia, neuropathic pain, diabetic neuropathy, lower back pain, multiple sclerosis, cystitis, rheumatoid arthritis, Crohn's Disease, inflammatory bowel disease (IBD), psoriasis, psoriatic arthritis, ankylosing spondylitis, vasculitis, myositis, Muckle-Wells Syndrome, Familial Mediterranean Fever (FMF) and a number of other clinical maladies. This compound is a mixed CB1- and CB2-agonist, which has obtained clinical evidence of efficacy and safety in normal healthy volunteers and patients with refractory, traumatic neuropathic pain. Administration of the compound, however, is associated with adverse events related to Cₘₐₓ, as we have discovered, and a short duration of efficacy.

Accordingly, there is a need for new compositions and methods of using ajulemic acid and other THC derivatives that maximize therapeutic benefit and minimize adverse events.

### SUMMARY OF THE INVENTION

The invention is based on the discovery of pharmacological levels of THC derivatives that optimize therapeutic or prophylactic effect while minimizing adverse events. The invention provides novel dosage forms and novel methods of administering THC derivatives of formula I:

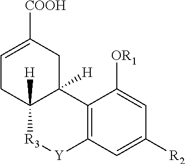

wherein R₁ is hydrogen, COCH₃ or COCH₂CH₃; R₂ is a branched C₅-C₁₂ alkyl group that may optionally have a terminal aromatic ring (e.g., phenyl), or optionally a branched OCHCH₃(CH₂)ₘCH₃ alkyl group that may have a terminal aromatic ring (e.g., phenyl), wherein m is 0 to 7; R₃ is hydrogen, a C₁₋₈ alkyl or a C₁₋₈ alkanol group; and Y is nil or a bridging group of NH or oxygen, provided that where Y is oxygen and R₂ is a branched C₅₋₁₂ alkyl, R₃ is not CHCH₃, and pharmaceutically acceptable salts, esters (e.g., a C₁₋₁₅ alkyl ester of the carboxy group), or solvates thereof. An exemplary compound of formula I is ajulemic acid. The THC derivative, or compositions containing the derivative, can be used, e.g., in the treatment of CB1/CB2 associated disease, such as pain and inflammation in osteoarthritis, systemic lupus erythematosus, post herpetic neuralgia, neuropathic pain, diabetic neuropathy, lower back pain, multiple sclerosis, cystitis, rheumatoid arthritis, Crohn's Disease, inflammatory bowel disease (IBD), psoriasis, psoriatic arthritis, ankylosing spondylitis, vasculitis, myositis, Muckle-Wells Syndrome, and Familial Mediterranean Fever (FMF). Other potential therapeutic indications include neck pain, shoulder pain, temporomandibular joint pain, chronic pelvic pain, complex regional pain syndrome, musculoskeletal pain, osteoarthritis pain, rheumatoid arthritis pain, fibromyalgia pain, cancer pain, chemotherapy-induced neuropathy pain, neuropathy pain secondary to tumor-infiltration, trigeminal neuralgia pain, postherpetic neuralgia pain, phantom limb pain, HIV sensory neuropathy pain, HIV myelopathy pain, peripheral diabetic neuropathy pain, gout pain, pain with irritable bowel syndrome, bladder pain, central post-stroke pain, multiple sclerosis pain, spinal cord injury pain, sciatic nerve pain, post ischemic myelopathy pain, and orthopedic pain. The THC derivative, e.g., ajulemic acid, may be employed either alone or in combination with other agents, such as analgesics or anti-inflammatory agents, which are well known in the art.

In one aspect, the invention provides a therapeutically or prophylactically effective amount of a compound of formula I:

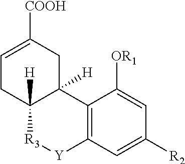

wherein R₁ is hydrogen, COCH₃ or COCH₂CH₃; R₂ is a branched C₅-C₁₂ alkyl group that may optionally have a terminal aromatic ring, or optionally a branched OCHCH₃(CH₂)ₘCH₃ alkyl group that may have a terminal aromatic ring, wherein m is 0 to 7; R₃ is hydrogen, a C₁₋₈ alkyl group or a C₁₋₈ alkanol group; and Y is nil or a bridging group of NH or oxygen, provided that where Y is oxygen and R₂ is a branched C₅₋₁₂ alkyl, R₃ is not CHCH₃, or a pharmaceutically acceptable salt, ester, or solvate thereof and a pharmaceutically acceptable carrier. In certain embodiments, administration of the composition results in a substantial reduction in an adverse event or risk thereof, such as one or more of dizziness, dry mouth, disorientation, euphoria, headache, nausea, pallor, somnolence, vomiting, tremor, abnormal feeling, tachycardia, fatigue, feeling drunk, paraesthesia, muscle spasms, muscle tightness, disturbance in attention, déjà vu, altered mood, anorexia, and cardiovascular events such as orthostatic hypotension, or QTc prolongation. In one embodiment, the therapeutically or prophylactically effective amount is released over at least 8 hours or 12 hours. The therapeutically or prophylactically effective amount is, for example, 5 to 240 mg, 5 to 180 mg, 5 to 120 mg, or 20 to 120 mg. The maximum exposure to the compound of formula I, based on AUC₀₋₂₄ₕᵣ, is, for example, less than 36,000 ng-hr/ml. The maximum exposure to the compound of formula I, based on AUC₀₋₂₄ₕᵣ, is, for example, less than 25,000 ng-hr/ml. The minimum exposure to the compound of formula I, based on AUC₀₋₂₄ₕᵣ, is, for example 5,000 ng-hr/ml. The maximum exposure to the compound of formula I, based on Cₘₐₓ, is, for example, less than 2500 ng/ml. The maximum exposure to the compound of formula I, based on Cₘᵢₙ, is, for example, less than 1200 ng/ml. In preferred embodiments, the therapeutic or prophylactic effect is maintained by administration of less than four doses per day of the pharmaceutical composition. The minimum exposure to the compound of formula I, based on Cₘᵢₙ, is, for example, 100 to 500 ng/ml. The therapeutically or prophylactically effective amount is, for example, 5 to 60 mg, the maximum exposure to the compound of formula I based on AUC₀₋₂₄ is, for example, 5,000 to 30,000 ng-hr/ml, based on Cₘₐₓ is, for example, less than 1200 ng/ml, and based on Cₘᵢₙ, is, for example, 100 to 500 ng/ml. In certain embodiments, Tₘₐₓ is less than 4 hours. The compound of formula I in any of the pharmaceutical compositions is, for example, ajulemic acid. The composition may be formulated to provide a therapeutically or prophylactically effective amount when administered once a day, twice a day, or three times a day.

In another aspect, the invention provides a method of delivering a compound of formula I by administering a pharmaceutical composition as described herein to a subject in need thereof.

The invention also provides a method of administering a therapeutically or prophylactically effective amount of a compound of formula I:

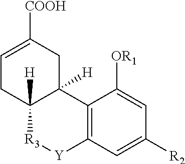

wherein R₁ is hydrogen, COCH₃ or COCH₂CH₃; R₂ is a branched C₅-C₁₂ alkyl group that may optionally have a terminal aromatic ring, or optionally a branched OCHCH₃(CH₂)ₘCH₃ alkyl group that may have a terminal aromatic ring, wherein in is 0 to 7; R₃ is hydrogen, a C₁₋₈ alkyl group or a C₁₋₈ alkanol group; and Y is nil or a bridging group of NH or oxygen, provided that where Y is oxygen and R₂ is a branched C₅₋₁₂ alkyl, R₃ is not CHCH₃, or a pharmaceutically acceptable salt, ester, or solvate thereof, wherein

(a) administration of the compound of formula I results in a substantial reduction in an adverse event or risk thereof; and/or

(b) the compound of formula I is administered in a sustained release formulation.

In these methods, (i) the maximum exposure to the compound of formula I, based on AUC₀₋₂₄ₕᵣ, is, for example, less than 36,000 ng-hr/ml;

(ii) the minimum exposure to the compound of formula I, based on AUC₀₋₂₄ₕᵣ, is, for example, 5000 ng-hr/ml;

(iii) the maximum exposure to the compound of formula I, based on Cₘₐₓ, is, for example, less than 2500 ng/ml;

(iv) the maximum exposure to the compound of formula I, based on Cₘᵢₙ, is, for example, less than 1200 ng/ml; and/or

(v) the minimum exposure to the compound of formula I, based on Cₘᵢₙ, is, for example, 100 to 500 ng/ml.

The therapeutically or prophylactically effective amount per dosage form is, for example, 5 to 60 mg, the maximum exposure to the compound of formula I based on AUC₀₋₂₄ is, for example, 5,000 to 30,000 ng-hr/ml, based on Cₘₐₓ is, for example, less than 1200 ng/ml, and based on Cₘᵢₙ is, for example, 100 to 500 ng/ml. Tₘₐₓ is, for example, less than 4 hours. The adverse event is, for example, one or more of dizziness, dry mouth, disorientation, euphoria, headache, nausea, pallor, somnolence, vomiting, tremor, abnormal feeling, tachycardia, fatigue, feeling drunk, paraesthesia, muscle spasms, muscle tightness, disturbance in attention, déjà vu, altered mood, anorexia, and cardiovascular events such as orthostatic hypotension, or QTc prolongation.

In any of the methods of administering a compound of formula I, the administering is, for example, for the treatment or prevention of a CB1/CB2 associated disease or for the treatment or prevention of pain or inflammation. The subject can be diagnosed with pain or inflammation or another CB1/CB2 associated disease. Exemplary conditions for treatment or prevention are selected from the group consisting of osteoarthritis, systemic lupus erythematosus, post herpetic neuralgia, neuropathic pain, diabetic neuropathy, lower back pain, multiple sclerosis, cystitis, rheumatoid arthritis, Crohn's Disease, inflammatory bowel disease (IBD), psoriasis, psoriatic arthritis, ankylosing spondylitis, vasculitis, myositis, Muckle-Wells Syndrome, and Familial Mediterranean Fever (FMF). Additional conditions include neck pain, shoulder pain, temporomandibular joint pain, chronic pelvic pain, complex regional pain syndrome, musculoskeletal pain, osteoarthritis pain, rheumatoid arthritis pain, fibromyalgia pain, cancer pain, chemotherapy-induced neuropathy pain, neuropathy pain secondary to tumor-infiltration, trigeminal neuralgia pain, postherpetic neuralgia pain, phantom limb pain, HIV sensory neuropathy pain, HIV myelopathy pain, peripheral diabetic neuropathy pain, gout pain, pain with irritable bowel syndrome, bladder pain, central post-stroke pain, multiple sclerosis pain, spinal cord injury pain, sciatic nerve pain, post ischemic myelopathy pain, orthopedic pain, pain from cardiovascular disease, breast pain, psoriasis, eczema, dermatitis, burn, peripheral neuropathic and central neuropathic pain, chronic pain, crush injury and trauma induced pain, cerebrovascular and vascular pain, sickle cell disease pain, orofacial and facial pain including dental, surgical incision related pain, visceral pain, soft tissue inflammatory pain, reflex sympathetic dystrophy, and pain resulting from kidney stones or urinary tract infection.

In any of the methods, the compound of formula I can be administered to the subject over a period of time of at least 8, 10, 14, 21, 28, 60, 90, 120, or 365 days either continuously, intermittently or with some interruptions. In these embodiments, the subject may receive a dosage on a daily basis. The compound of formula I can be administered once, twice, or thrice daily. In other embodiments, the compound of formula I is administered at least once daily for at least 7, 14, or 30 days. The amount of the compound of formula I administered is, for example, from 5-240 mg/day, 5-180 mg/day, 5-120 mg/day, or 20-120 mg/day. In any of the methods, a preferred compound of formula I is ajulemic acid.

The invention also features a compound of formula I:

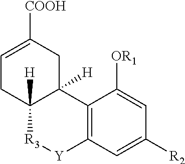

wherein R₁ is hydrogen, COCH₃ or COCH₂CH₃; R₂ is a branched C₅-C₁₂ alkyl group that may optionally have a terminal aromatic ring, or optionally a branched OCHCH₃(CH₂)ₘCH₃ alkyl group that may have a terminal aromatic ring, wherein m is 0 to 7; R₃ is hydrogen, a C₁₋₈ alkyl group or a C₁₋₈ alkanol group; and Y is nil or a bridging group of NH or oxygen, provided that where Y is oxygen and R₂ is a branched C₅₋₁₂ alkyl, R₃ is not CHCH₃, or a pharmaceutically acceptable salt, ester, or solvate thereof for use in a method of treating or preventing a CB1/CB2 associated disease, wherein

(a) administration of the compound of formula I results in a substantial reduction in an adverse event or risk thereof; and/or

(b) the compound of formula I is administered in a sustained release formulation.

In use, (i) the maximum exposure to the compound of formula I, based on AUC₀₋₂₄ₕᵣ, is, for example, less than 36,000 ng-hr/ml;

(ii) the minimum exposure to the compound of formula I, based on AUC₀₋₂₄ₕᵣ, is, for example, 5000 ng-hr/ml;

(iii) the maximum exposure to the compound of formula I, based on Cₘₐₓ, is, for example, less than 2500 ng/ml;

(iv) the maximum exposure to the compound of formula I, based on Cₘᵢₙ, is, for example, less than 1200 ng/ml; and/or

(v) the minimum exposure to the compound of formula I, based on Cₘᵢₙ, is, for example, 100 to 500 ng/ml.

The therapeutically or prophylactically effective amount is, for example, 5 to 60 mg, the maximum exposure to the compound of formula I based on AUC₀₋₂₄ is, for example, 5000 to 30,000 ng-hr/ml, based on Cₘₐₓ is, for example, less than 1200 ng/ml, and based on Cₘᵢₙ is, for example, 100 to 500 ng/ml. Tₘₐₓ is preferably less than 4 hours. The subject may be diagnosed with pain or inflammation or another CB1/CB2 associated disease. The use is, for example, for the treatment or prevention of pain or inflammation.

Exemplary conditions for treatment or prevention include osteoarthritis, systemic lupus erythematosus, post herpetic neuralgia, neuropathic pain, diabetic neuropathy, lower back pain, multiple sclerosis, cystitis, rheumatoid arthritis, Crohn's Disease, inflammatory bowel disease (IBD), psoriasis, psoriatic arthritis, ankylosing spondylitis, vasculitis, myositis, Muckle-Wells Syndrome, and Familial Mediterranean Fever (FMF). Additional conditions include neck pain, shoulder pain, temporomandibular joint pain, chronic pelvic pain, complex regional pain syndrome, musculoskeletal pain, osteoarthritis pain, rheumatoid arthritis pain, fibromyalgia pain, cancer pain, chemotherapy-induced neuropathy pain, neuropathy pain secondary to tumor-infiltration, trigeminal neuralgia pain, postherpetic neuralgia pain, phantom limb pain, HIV sensory neuropathy pain, HIV myelopathy pain, peripheral diabetic neuropathy pain, gout pain, pain with irritable bowel syndrome, bladder pain, central post-stroke pain, multiple sclerosis pain, spinal cord injury pain, sciatic nerve pain, post ischemic myelopathy pain, orthopedic pain, pain from cardiovascular disease, breast pain, psoriasis, eczema, dermatitis, burn, peripheral neuropathic and central neuropathic pain, chronic pain, crush injury and trauma induced pain, cerebrovascular and vascular pain, sickle cell disease pain, orofacial and facial pain including dental, surgical incision related pain, visceral pain, soft tissue inflammatory pain, reflex sympathetic dystrophy, and pain resulting from kidney stones or urinary tract infection.

In any of the uses, the compound of formula I can be administered to the subject over a period of time of at least 8, 10, 14, 21, 28, 60, 90, 120, or 365 days either continuously, intermittently or with some interruptions. In these embodiments, the subject may receive a dosage on a daily basis. The compound of formula I can be administered once, twice, or thrice daily. In other embodiments, the compound of formula I is administered at least once daily for at least 7, 14, or 30 days. The amount of the compound of formula I administered is, for example, from 5-240 mg/day, 5-180 mg/day, 5-120 mg/day, or 20-120 mg/day. In any of the methods, a preferred compound of formula I is ajulemic acid.

In an embodiment, any method or use includes selecting a dosage form, and administration regimen, on the basis that the maximum (and optionally the minimum) levels of THC derivative, e.g., ajulemic acid, referred to above will not be exceeded.

In an embodiment, the subject in the method or use is in need of the blood levels of THC derivative, e.g., ajulemic acid, referred to above.

In some embodiments, the invention provides specific dosage forms. In one embodiment, the form is an oral dosage form such as a tablet or capsule or enteric coated tablet or osmotic release capsule or unique combination of excipients formulated in such a way as to deliver over a 24 hour period not more than 240 mg and not less than 5 mg of a THC derivative, e.g., ajulemic acid, more preferably not more than 180 mg and not less than 15 mg (e.g., from 120 mg to 30 mg). In some embodiments, the dosage form is a topical patch, gel, ointment, or cream.

In some embodiments, the dosage form includes an additional agent or is provided together with a second dosage form which includes the additional agent. Exemplary additional agents include an analgesic agent such as an NSAID or opiate, or other anti-inflammatory agent.

In certain embodiments, the dosage form is a tablet coated with a semi-permeable coating. In certain embodiments, the tablet comprises two layers, a layer containing the THC derivative, e.g., ajulemic acid, and a second layer referred to as a “push” layer. The semi-permeable coating is used to allow a fluid (e.g., water) to enter the tablet and erode a layer or layers. In certain embodiments, the sustained release dosage form further comprises a laser hole drilled in the center of the coated tablet. The THC derivative, e.g., ajulemic acid, containing layer contains the THC derivative, a disintegrant, a viscosity enhancing agent, a binding agent, and an osmotic agent. The push layer includes a disintegrant, a binding agent, an osmotic agent, and a viscosity enhancing agent.

In certain embodiments, the dosage form is a tablet including a biocompatible matrix and a THC derivative, e.g., ajulemic acid. The sustained release dosage form may also include a hard-shell capsule containing bio-polymer microspheres containing a THC derivative, e.g., ajulemic acid. The biocompatible matrix and bio-polymer microspheres each contain pores for drug release and delivery. These pores are formed by mixing the biocompatible matrix of bio-polymer microsphere with a pore forming agent. Each biocompatible matrix of bio-polymer microsphere is made up of a biocompatible polymer or mixture of biocompatible polymers. The matrix and microspheres can be formed by dissolving the biocompatible polymer and THC derivative, e.g., ajulemic acid, in a solvent and adding a pore forming agent (e.g., a volatile salt). Evaporation of the solvent and pore forming agent provides a matrix or microsphere containing the THC derivative.

In certain embodiments, the sustained release dosage form is a tablet containing a THC derivative, e.g., ajulemic acid, and one or more polymers. The tablet can be prepared by compressing the THC derivative, e.g., ajulemic acid, and one or more polymers. In some embodiments, the one or more polymers may comprise a hygroscopic polymer formulated with THC derivative, e.g., ajulemic acid. Upon exposure to moisture, the tablet dissolves and swells. This swelling allows the sustained release dosage form to remain in the upper GI tract. The swelling rate of the polymer mixture can be varied using different grades of polyethylene oxide.

In certain embodiments, the sustained release dosage form comprises a capsule further comprising particle cores coated with a suspension of THC derivative, e.g., ajulemic acid, and a binding agent which is subsequently coated with a polymer. The polymer may be a rate-controlling polymer. In general, the delivery rate of the rate-controlling polymer is determined by the rate at which the active agent is dissolved.

In another aspect, the invention features, a kit, comprising a plurality of unit dosage forms of THC derivative, e.g., ajulemic acid. In an embodiment the kit contains at least 90, 100, 500, 1,000, 1,500 or 2,000 mg of THC derivative, e.g., ajulemic acid. In an embodiment the kit includes at least 10, 20, 30, 40, 50, or 100 unit dosage forms, e.g., each of which contain the same amount of THC derivative, e.g., ajulemic acid. In an embodiment the kit includes at least 1, 2, 5, 10, 20, 30, 40, 50, or 100 unit dosage forms, e.g., each of which contains at least 10 mg, e.g., at least 15, 20, 30, 40, 50, 60, 70, 80, 90, 100, 110, or 120 mg of THC derivative, e.g., ajulemic acid.

Other features and advantages will be apparent from the following description, the examples, and the claims.

### DEFINITIONS

By “AUC₀₋₂₄” is meant the amount of THC derivative, e.g., ajulemic acid, delivered to a subject in a 24 hour period, measured in the plasma in units of ng-hr/ml. In determining AUC₀₋₂₄, the THC derivative, e.g., ajulemic acid, may be administered after at least a 10 hour fast. AUC₀₋₂₄ may also be determined on the basis of the average plasma values in a population of subjects, e.g., 10 or more subjects to which the THC derivative is administered under the same circumstances.

By “CB1/CB2 associated disease” is meant a disease that can be treated or prevented by administration of an agonist to the CB1 and/or CB2 receptor.

By “Cₘₐₓ” is meant the maximum concentration of THC derivative, e.g., ajulemic acid, reached in the plasma of a subject during a 24 hour period. In determining, Cₘₐₓ, the THC derivative, e.g., ajulemic acid, may be administered initially after at least a 10 hour fast. Cₘₐₓ may also be determined on the basis of the average plasma values in a population of subjects, e.g., 10 or more subjects to which the THC derivative is administered under the same circumstances.

By “Cₘᵢₙ” is meant the minimum concentration of THC derivative, e.g., ajulemic acid, reached in the plasma of a subject after administration of the THC derivative under a dosing regimen of between 1 and three doses per day and lasting at least three days, e.g., at least 7 or 14 days. Cₘᵢₙ may also be determined on the basis of the average plasma values in a population of subjects, e.g., 10 or more subjects to which the THC derivative is administered under the same circumstances.

By “pharmaceutically acceptable carrier, adjuvant, or vehicle” is meant a carrier, adjuvant, or vehicle that may be administered to a patient, together with a THC derivative, e.g., ajulemic acid, of this invention, and which does not destroy the pharmacological activity thereof and is nontoxic when administered in doses sufficient to deliver a therapeutic amount of the THC derivative.

As used herein, an amount of a THC derivative, e.g., ajulemic acid, effective to prevent a disorder, or “a prophylactically effective amount” refers to an amount effective, upon single- or multiple-dose administration to the subject, in preventing or delaying the occurrence of the onset or recurrence of a disorder or a symptom of the disorder.

By “subject” is meant human and non-human animals. Exemplary human subjects include a human patient having a disorder, e.g., a disorder described herein or a normal subject. The term “non-human animals” of the invention includes all vertebrates, e.g., non-mammals (such as chickens, amphibians, reptiles) and mammals, such as non-human primates, domesticated and/or agriculturally useful animals, e.g., sheep, dog, cat, cow, pig, etc. In an embodiment the animal is other than a rodent, e.g., a rat or mouse. In certain embodiments, the subject is a human under the age of 65.

By “substantial reduction” in an adverse event or risk thereof is meant a reduction by at least 10%, e.g., at least 20%, 30%, 40%, 50%, 60%, 70%, 80%, 90%, or even 95% of one or more adverse events in the population treated compared to treatment with the same amount of THC derivative, e.g., ajulemic acid, in an immediate release formulation or a reduction by at least 10%, e.g., at least 20%, 30%, 40%, 50%, 60%, 70%, 80%, 90%, or even 95% in the extent of one or more adverse events in a subject treated compared to treatment with the same amount of THC derivative, e.g., ajulemic acid, in an immediate release formulation.

By “Tₘₐₓ” is meant the time required to reach Cₘₐₓ after administration of a THC derivative, e.g., ajulemic acid, to a subject. In determining, Tₘₐₓ, the THC derivative, e.g., ajulemic acid, may be administered initially after at least a 10 hour fast. Tₘₐₓ may also be determined on the basis of the average plasma values in a population of subjects, e.g., 10 or more subjects to which the THC derivative is administered under the same circumstances.

As used herein, an amount of a THC derivative, e.g., ajulemic acid, effective to treat a disorder, or a “therapeutically effective amount” refers to an amount effective, upon single or multiple dose administration to a subject to achieve treatment.

As used herein, the term “treat” or “treatment” is defined as the administration of a THC derivative, e.g., ajulemic acid, by any route, e.g., orally, to a subject. The THC derivative, e.g., ajulemic acid, can be administered alone or in combination with a second compound. The subject can have a disorder, a symptom of a disorder, or a predisposition toward a disorder. Treatment can result in one or more of curing, healing, alleviating, relieving, altering, remedying, ameliorating, improving or affecting the disorder, one or more symptoms of the disorder or the predisposition toward the disorder. In an embodiment the treatment prevents at least one symptom of the disorder or to delays onset of at least one symptom of the disorder. The effect is beyond what is seen in the absence of treatment.

The term “pharmaceutically acceptable salt,” as used herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharm. Sci. 66(1):1-19, 1977 and in Pharmaceutical Salts: Properties, Selection, and Use, P. H. Stahl and C. G. Wermuth (eds.), Wiley-VCH, 2008. The salts can be prepared in situ during the final isolation and purification of the compounds of the invention or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecyl sulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “pharmaceutically acceptable solvate,” as used herein, means a THC derivative, e.g., ajulemic acid, wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

By “THC derivative” is meant a compound of formula I, e.g., ajulemic acid.

### BRIEF DESCRIPTION OF THE DRAWINGS

FIG. 1 is a graph of the dose proportionality of Cₘₐₓ and AUC₀₋₂₄ in a single dose study in human subjects of immediate release ajulemic acid.

FIG. 2 is a graph of the concentration of ajulemic acid as a function of time in a multiple ascending dose study in human subjects of ajulemic acid administered in an immediate release formulation three times a day (6-6-12 hours). For the 80<=60 mg data, the first four doses were 80 mg and all remaining doses were 60 mg.

FIG. 3 is a graph showing exemplary dissolution data of sustained released tablets.

### DETAILED DESCRIPTION OF THE INVENTION

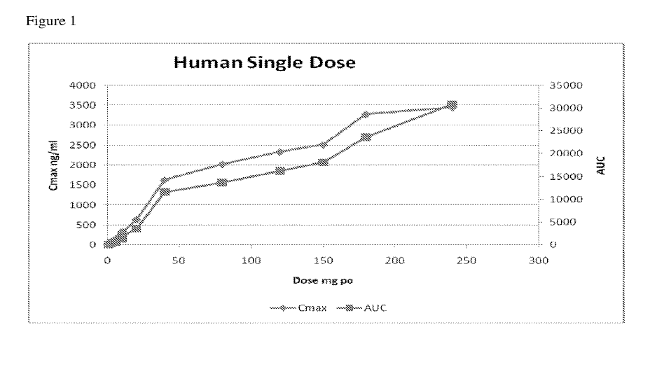

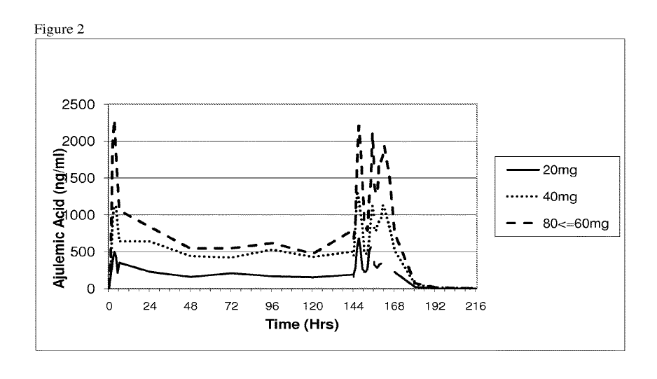

Administration of immediate release formulations of the THC derivative ajulemic acid may result in adverse events. Representative pharmacokinetic data for administration of ajulemic acid in immediate release form is shown in FIG. 1 (single ascending dose) and 2 (multiple ascending dose). The data in FIG. 2 are based on greater sampling days 1 and 7. In the single dose study, adverse events were observed when 80 mg or higher was administered. In the multiple dose study, adverse events were observed when 40, 60, and 80 mg was administered three times a day. In the single ascending dose study in fasted subjects, the most frequently occurring adverse events were dizziness (33.3%); nausea (20.8%); vomiting (14.6%); pallor (12.5%); dry mouth; headache; somnolence; tremor; and disorientation (10.4%). Other adverse events include tachycardia, fatigue, feeling drunk, paraesthesia, and euphoric mood (6.3%); and muscle spasms, muscle tightness, disturbance in attention, deja vu, and altered mood (4.2%). In the multiple ascending dose study, the most frequently occurring adverse events were dizziness (38.9%); nausea and somnolence (16.7%); and dry mouth, fatigue, feeling abnormal, anorexia, inappropriate affect, and orthostatic hypotension (11.1%).

The present invention provides methods and compositions that provide a therapeutically or prophylactically effective amount of a THC derivative, e.g., ajulemic acid, preferably resulting in a substantial reduction in adverse events or risk of adverse events. The occurrence of the adverse events typically coincides with the time at which the maximum concentration (Cₘₐₓ) of THC derivative is present in the blood after administration of the immediate release form. In certain embodiments, the invention provides sustained release compositions and methods that deliver a therapeutically or prophylactically effective amount of a THC derivative, e.g., ajulemic acid, equivalent to an immediate release formulation but with a lower maximum concentration and concurrent reduction in adverse events. The present invention also provides compositions and methods that extend the duration of therapeutic or prophylactic effect of a THC derivative, e.g., ajulemic acid, compared to immediate release formulations.

The present invention relates to derivatives of tetrahydrocannabinol of formula (I):

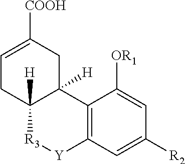

wherein R₁ is hydrogen, COCH₃ or COCH₂CH₃; R₂ is a branched C₅-C₁₂ alkyl group that may optionally have a terminal aromatic ring (e.g., phenyl), or optionally a branched OCHCH₃(CH₂)ₘCH₃ alkyl group that may have a terminal aromatic ring (e.g., phenyl), wherein m is 0 to 7; R₃ is hydrogen, a C₁₋₈ alkyl or a C₁₋₈ alkanol group; and Y is nil or a bridging group of NH or oxygen, provided that where Y is oxygen and R₂ is a branched C₅₋₁₂ alkyl, R₃ is not CHCH₃, and pharmaceutically acceptable salts, esters (e.g., a C₁₋₁₅ alkyl ester of the carboxy group), or solvates thereof. A preferred compound is ajulemic acid. Methods of synthesizing compounds of formula I are known in the art, e.g., as described in U.S. Pat. No. 5,338,753. The THC derivatives, e.g., ajulemic acid, can be used to treat or prevent CB1/CB2 associated diseases, such as pain and inflammation and other disorders described herein. In certain embodiments, the dosage, when administered to a subject (e.g., a mammal such as a human), results in no more than a maximum exposure based on AUC₀₋₂₄ₕᵣ of <36,000 ng-hr/ml and more preferably <25,000 ng-hr/ml and ideally between 5000 and 22,000 ng-hr/ml; based on Cₘₐₓ of <3600 ng/ml, more preferably <2500 ng/ml, and ideally between 200-2000 ng/ml; and based on Cₘᵢₙ<1500 ng/ml, more preferably <1200 ng/ml, and ideally between 100-500 ng/ml.

### Unit Dosage Formulations

The THC derivative, e.g., ajulemic acid, can be administered to a subject using a number of different dosage forms. The dosage forms of this invention may be administered orally, parenterally, rectally, buccally, vaginally, or via an implanted reservoir. The term parenteral as used herein includes subcutaneous, intracutaneous, intravenous, intramuscular, intraarticular, intraarterial, intrasynovial, intrasternal, intrathecal, intralesional and intracranial injection or infusion techniques. Exemplary dosage forms include oral dosage forms (e.g., a tablet or capsule).

The pharmaceutical compositions of this invention may contain any conventional non-toxic pharmaceutically-acceptable carriers, adjuvants, or vehicles. In some cases, the pH of the formulation may be adjusted with pharmaceutically acceptable acids, bases or buffers to enhance the stability of the formulated compound or its delivery form.

In some embodiments, the THC derivative, e.g., ajulemic acid, is formulated into a dosage form wherein a single dosage is from 5 to 240 mg, e.g., 5 to 180 mg, 5 to 120 mg, 5 to 60 mg, or 20 to 120 mg once daily. Alternatively, the THC derivative may be formulated for delivery twice or three times a day, e.g., containing from 5 to 80 mg, e.g., 5 to 40 mg up to 3 times daily.

In some embodiments, the THC derivative, e.g., ajulemic acid, is formulated into a dosage form wherein a single dosage is from 0.1 to 0.8 mg/kg weight of the subject, which can be administered up to three times a day. Alternatively, 0.3 to 2.4 mg/kg weight of the subject can be administered once daily, e.g., in sustained release form, or 0.15 to 1.2 mg/kg of weight of the subject can be administered twice daily.

In some embodiments, the THC derivative, e.g., ajulemic acid, is formulated into a dosage form wherein the dosage form, when administered to a subject has an exposure based on AUC₀₋₂₄ₕᵣ of 5000-36,000 ng-hr/ml, e.g., less than 22,000, 25,000, or 30,000 ng-hr/ml; Cₘₐₓ<2500 ng/ml, e.g., <1200, 1500, or 2000 ng/ml; Cₘₐₓ>100, 200, or 300 ng/ml and less than 2000 or 2500 ng/ml; and Cₘᵢₙ<1200 or 1500 ng/ml, e.g., 50-1000 ng/ml or 100-500 ng/ml. Such dosage forms may include between 5 to 60 mg of the THC derivative, e.g., ajulemic acid, and may be intended for once, twice, or three times daily administration. Preferably, the dosage form has a Tₘₐₓ of less than 4 hr. In an embodiment, a sustained release formulation of 120 mg of THC derivative, e.g., ajulemic acid, has a maximum exposure based on AUC₀₋₂₄ₕᵣ of <25,000 ng-hr/ml.

In an embodiment, the administration regimen including dosage and timing ensures that the maximum (and optionally the minimum) levels of THC derivative, e.g., ajulemic acid, referred to above will not be exceeded. In an embodiment, the subject is in need of the blood levels of THC derivative, e.g., ajulemic acid, referred to above to achieve maximum efficacy and minimum side effects.

The THC derivative, e.g., ajulemic acid, will be formulated with a pharmaceutically acceptable carrier or adjuvant. Pharmaceutically acceptable carriers, adjuvants, and vehicles that may be used in the dosage forms of this invention include, but are not limited to, ion exchangers, alumina, aluminum stearate, lecithin, self-emulsifying drug delivery systems (SEDDS) (such as d-α-tocopherol polyethyleneglycol 1000 succinate), surfactants used in pharmaceutical dosage forms (such as Tweens or other similar polymeric delivery matrices), serum proteins (such as human serum albumin), buffer substances (such as phosphates), glycine, sorbic acid, potassium sorbate, partial glyceride mixtures of saturated vegetable fatty acids, water, salts or electrolytes (such as protamine sulfate, disodium hydrogen phosphate, potassium hydrogen phosphate, or sodium chloride), zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used in formulations of the THC derivative, e.g., ajulemic acid. Additional excipients may be found in the Handbook of Pharmaceutical Excipients, R. C. Rowe, et al., Pharmaceutical Press, 2009, In some cases, the pH of the formulation may be adjusted with pharmaceutically acceptable acids, bases or buffers to enhance the stability of the formulated compound or its delivery form.

The dosage forms of this invention may be orally administered in any orally acceptable dosage form including, but not limited to, capsules, tablets, emulsions, aqueous suspensions, dispersions, and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase combined with emulsifying and/or suspending agents. If desired, certain sweetening and/or flavoring and/or coloring agents may be added.

In some embodiments, unit dosage formulations are compounded for sustained release, though unit dosage formulations compounded for immediate release of one or more agents are also contemplated. In some embodiments, multiple agents are formulated in a single unit dose such that the agents are released from the dosage at different times. The sustained release formulation may be designed to deliver a THC derivative, e.g., ajulemic acid, over a period of at least 6, 8, or 12 hours.

In certain embodiments, the dosage form is a capsule wherein the capsule includes a mixture of material to provide the desired sustained release. In certain embodiments, the dosage form is a tablet coated with a semi-permeable coating. In certain embodiments, the tablet includes two layers, a layer containing the THC derivative, e.g., ajulemic acid, and a second layer referred to as a “push” layer. The semi-permeable coating is used to allow a fluid (e.g., water) to enter the tablet and erode a layer or layers. In certain embodiments, the sustained release dosage form further comprises a laser hole drilled in the center of the coated tablet. The THC derivative, e.g., ajulemic acid, containing layer includes the THC derivative, a disintegrant, a viscosity enhancing agent, a binding agent and an osmotic agent. The push layer includes a disintegrant, a binding agent, an osmotic agent and a viscosity enhancing agent.

In certain embodiments, the dosage form comprises a tablet comprising a biocompatible matrix and a THC derivative, e.g., ajulemic acid. The sustained release dosage form may also comprise a hard-shell capsule containing bio-polymer microspheres containing a THC derivative, e.g., ajulemic acid. The biocompatible matrix and bio-polymer microspheres each contain pores for drug release and delivery. These pores are formed by mixing the biocompatible matrix of bio-polymer microsphere with a pore forming agent. Each biocompatible matrix of bio-polymer microsphere is made up of a biocompatible polymer or mixture of biocompatible polymers. The matrix and microspheres can be formed by dissolving the biocompatible polymer and active agent (compound described herein) in a solvent and adding a pore forming agent (e.g., a volatile salt). Evaporation of the solvent and pore forming agent provides a matrix or microsphere containing the THC derivative, e.g., ajulemic acid.

In certain embodiments, the sustained release dosage form is a tablet, wherein the tablet contains a THC derivative, e.g., ajulemic acid, and one or more polymers. The tablet can be prepared by compressing the THC derivative, e.g., ajulemic acid, and one or more polymers. In some embodiments, the one or more polymers may include a hygroscopic polymer. Upon exposure to moisture, the tablet dissolves and swells. This swelling allows the sustained release dosage form to remain in the upper GI tract. The swelling rate of the polymer mixture can be varied using different grades of polyethylene oxide.

In certain embodiments, the sustained release dosage form is a capsule further including particle cores coated with a suspension of a THC derivative, e.g., ajulemic acid, and a binding agent which is subsequently coated with a polymer. The polymer may be a rate-controlling polymer. In general, the delivery rate of the rate-controlling polymer is determined by the rate at which the active agent is dissolved. Examples of capsules include but are not limited to gelatin capsules, HPMC, hard shell, soft shell, or any other suitable capsule for holding a sustained release mixture.

In an alternative embodiment, the THC derivative, e.g., ajulemic acid, is formulated using a biphasic controlled release delivery system, thereby providing prolonged gastric residence. For example, in some embodiments, the delivery system includes (1) an inner solid particulate phase formed of substantially uniform granules containing the THC derivative, and one or more hydrophilic polymers, one or more hydrophobic polymers and/or one or more hydrophobic materials such as one or more waxes, fatty alcohols and/or fatty acid esters, and (2) an outer solid continuous phase in which the above granules of inner solid particulate phase are embedded and dispersed throughout, the outer solid continuous phase including one or more hydrophobic polymers, one or more hydrophobic polymers and/or one or more hydrophobic materials such as one or more waxes, fatty alcohols and/or fatty acid esters, which may be compressed into tablets or filled into capsules. In some embodiments, the THC derivative is incorporated into polymeric matrices of hydrophilic polymers that swell upon imbibition of water to a size that is large enough to promote retention of the dosage form in the stomach during the fed mode.

The solvents used in the above sustained release dosage forms include, but are not limited to ethyl acetate, triacetin, dimethyl sulfoxide (DMSO), propylene carbonate, N-methylpyrrolidone (NMP), ethyl alcohol, benzyl alcohol, glycofurol, alpha-tocopherol, Miglyol 810, isopropyl alcohol, diethyl phthalate, polyethylene glycol 400 (PEG 400), triethyl citrate, and benzyl benzoate.

The viscosity modifiers used in the above sustained release dosage forms include, but are not limited to caprylic/capric triglyceride (Migliol 810), isopropyl myristate (IPM), ethyl oleate, triethyl citrate, dimethyl phthalate, benzyl benzoate, and various grades of polyethylene oxide.

The high viscosity liquid carrier used in the above sustained release dosage forms include, but are not limited to sucrose acetate isobutyrate (SAIB) and cellulose acetate butyrate (CAB) 381-20.

Examples of materials that make up preferred semi-permeable layers include, but are not limited to cellulosic polymers such as cellulose acetate, cellulose acylate, cellulose diacylate, cellulose triacylate, cellulose diacetate, cellulose triacetate or any mixtures thereof ethylene vinyl acetate copolymers, polyethylene, copolymers of ethylene, polyolefins including ethylene oxide copolymers (e.g., Engage®—Dupont Dow Elastomers), polyamides, cellulosic materials, polyurethanes, polyether blocked amides, and copolymers (e.g., PEBAX®, cellulosic acetate butyrate and polyvinyl acetate).

Examples of disintegrants that may be employed in the above sustained release dosage forms include but are not limited to croscarmellose sodium, crospovidone, sodium alginate or similar excipients.

Examples of binding agents that may be employed in the above sustained release dosage forms include but are not limited to hydroxyalkylcellulose, a hydroxyalkylalkylcellulose, or a polyvinylpyrrolidone.

Examples of osmotic agents that may be employed in the above sustained release dosage forms include but are not limited to sorbitol, mannitol, sodium chloride, or other salts.

Examples of biocompatible polymers employed in the above sustained release dosage forms include but are not limited to poly(hydroxyl acids), polyanhydrides, polyorthoesters, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polysiloxanes, poly(vinyl alcohols), poly (vinyl acetate), polystyrene, polyurethanes and co-polymers thereof, synthetic celluloses, polyacrylic acids, poly(butyric acid), poly(valeric acid), and poly(lactide-co-caprolactone), ethylene vinyl acetate, copolymers and blends thereof.

Examples of hygroscopic polymers that may be employed in the above sustained release dosage forms include but are not limited to polyethylene oxide (e.g., Polyox® with MWs from 4,000,000 to 10,000,000), cellulose, hydroxymethyl cellulose, hydroxyethyl-cellulose, crosslinked polyacrylic acids, and xanthum gum.

Examples of rate-controlling polymers the may be employed in the above sustained release dosage forms include but are not limited to polymeric acrylate, methacrylate lacquer or mixtures thereof, polymeric acrylate lacquer, methacrylate lacquer, an acrylic resin of a copolymer of acrylic and methacrylic acid esters, or an ammonium methacrylate lacquer with a plasticizer.

The dosage forms of this invention may also be administered in the form of suppositories for rectal administration. These compositions can be prepared by mixing a THC derivative, e.g., ajulemic acid, with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the dosage forms of this invention is useful when the desired treatment involves areas or organs readily accessible by topical application. Topically-transdermal patches are also included in this invention.

When the dosage forms of this invention include a combination of THC derivative, e.g., ajulemic acid, and one or more additional therapeutic or prophylactic agents, both the THC derivative and the additional agent should be present at dosage levels of between about 1 to 100%, and more preferably between about 5 to 95% of the dosage normally administered in a monotherapy regimen. The additional agents may be administered separately, as part of a multiple dose regimen, from the THC derivative, e.g., ajulemic acid. Alternatively, those agents may be part of a single dosage form, mixed together with the compounds of this invention in a single composition.

In some embodiments, the dosage form allows for immediate release of a portion of the THC derivative, e.g., ajulemic acid, and sustained release of the remainder of the THC derivative.

### Methods of Treatment or Prevention

A THC derivative, e.g., ajulemic acid, can be used in the treatment or prevention of a CB1/CB2 associated disease. Examples of such diseases include pain and inflammation, e.g., in osteoarthritis, systemic lupus erythematosus, post herpetic neuralgia, neuropathic pain, diabetic neuropathy, lower back pain, multiple sclerosis, cystitis, rheumatoid arthritis, Crohn's Disease, inflammatory bowel disease (IBD), psoriasis, psoriatic arthritis, ankylosing spondylitis, vasculitis, myositis, Muckle-Wells Syndrome, and Familial Mediterranean Fever (FMF). Other potential therapeutic indications include neck pain, shoulder pain, temporomandibular joint pain, chronic pelvic pain, complex regional pain syndrome, musculoskeletal pain, osteoarthritis pain, rheumatoid arthritis pain, fibromyalgia pain, cancer pain, chemotherapy-induced neuropathy pain, neuropathy pain secondary to tumor-infiltration, trigeminal neuralgia pain, postherpetic neuralgia pain, phantom limb pain, HIV sensory neuropathy pain, HIV myelopathy pain, peripheral diabetic neuropathy pain, gout pain, pain with irritable bowel syndrome, bladder pain, central post-stroke pain, multiple sclerosis pain, spinal cord injury pain, sciatic nerve pain, post ischemic myelopathy pain, orthopedic pain, pain from cardiovascular disease, breast pain, psoriasis, eczema, dermatitis, burn, peripheral neuropathic and central neuropathic pain, chronic pain, crush injury and trauma induced pain, cerebrovascular and vascular pain, sickle cell disease pain, orofacial and facial pain including dental, surgical incision related pain, visceral pain, soft tissue inflammatory pain, reflex sympathetic dystrophy, and pain resulting from kidney stones or urinary tract infection. Treatment or prevention methods may employ the THC derivative, e.g., ajulemic acid, alone or in combination with other agents, such as analgesics or anti-inflammatory agents, such as nonsteroidal anti-inflammatory drugs, opiate agonists, and salicylates. Other analgesics and anti-inflammatory agents are known in the art.

A THC derivative, e.g., ajulemic acid, may be administered as required to achieve the therapeutic or prophylactic effect desired. Typically, the THC derivative will be administered at least once daily, e.g., from one to three times daily. The THC derivative, e.g., ajulemic acid, can be administered to the subject over a period of time of at least 8, 10, 14, 21, 28, 60, 90, 120, or 365 days either continuously, intermittently or with some interruptions. In another embodiment, THC derivative, e.g., ajulemic acid, is administered at least once daily for at least 7, 14, or 30 days.

Treating a subject with a TEC derivative, e.g., ajulemic acid, can cause side effects as described herein and including dizziness, dry mouth, disorientation, euphoria, headache, nausea, pallor, somnolence, vomiting, tremor, abnormal feeling, tachycardia, fatigue, feeling drunk, paraesthesia, muscle spasms, muscle tightness, disturbance in attention, déjà vu, altered mood, anorexia, or cardiovascular events such as orthostatic hypotension, or QTc prolongation. The compositions and methods of the invention preferably substantially reduce these side effects.

### Kits

A dosage form described herein may be provided in a kit. The kit includes a THC derivative, e.g., ajulemic acid, and other ingredients, such as a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, and/or a second agent for treating a condition or disorder described herein. Alternatively, the other ingredients can be included in the kit, but in different compositions or containers than a THC derivative, e.g., ajulemic acid. In such embodiments, the kit can include instructions for admixing the derivative and the other ingredients, or for using the derivative together with the other ingredients.

In some embodiments, the components of the kit are stored under inert conditions (e.g., under Nitrogen or another inert gas such as Argon). In some embodiments, the components of the kit are stored under anhydrous conditions (e.g., with a desiccant). In some embodiments, the components are stored in a light blocking container such as an amber vial.

A dosage form described herein can be provided in any form, e.g., liquid, dried or lyophilized form. It is preferred that the THC derivative, such as ajulemic acid, be substantially pure and/or sterile. When the THC derivative, such as ajulemic acid, is provided in a liquid solution, the liquid solution preferably is an aqueous solution, with a sterile aqueous solution being preferred. When the THC derivative, such as ajulemic acid, is provided as a dried form, reconstitution generally is by the addition of a suitable solvent. The solvent, e.g., sterile water or buffer, can optionally be provided in the kit.

The kit can include one or more containers for the composition containing a dosage form described herein. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the dosage form is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampoules, foil packets, or blister packs, each containing a single unit dose of a dosage form described herein. The containers of the kits can be air tight, waterproof (e.g., impermeable to changes in moisture or evaporation), and/or light-tight.

The kit optionally includes a device suitable for use of the dosage form, e.g., a syringe, pipette, forceps, measured spoon, swab (e.g., a cotton swab or wooden swab), or any such device.

### EXAMPLES

### Example 1

### 20 mg Extended Release Tablet

This example describes an extended release matrix tablet that releases ajulemic acid over a period of time targeting about 80-90% of drug to be released in approximately 12 hours. The tablet matrix contains rate controlling polymer such as Methocel K100M Premium or Methocel K4M Premium, a diluent such as Prosolv Easytab, lactose monohydrate (FastFlo 316), optionally sodium lauryl sulfate, and a lubricant like magnesium stearate. Tables 1-4 provide the composition of exemplary tablets.

TABLE 1

| Item | mg/Tab | % (w/w) | Weight/Batch (g) |
|---|---|---|---|
| Ajulemic acid | 20.0 | 14.29 | 2.00 |
| Prosolve Easytab | 45.8 | 32.71 | 4.58 |
| Methocel K100M Premium DC | 37.8 | 27.00 | 3.78 |
| Lactose (FastFlo 316) | 36.0 | 25.00 | 3.50 |
| Magnesium stearate | 1.4 | 1.00 | 0.14 |
| Total | 140.0 | 100.00 | 14.00 |

TABLE 2

| Item | mg/Tab | % (w/w) | Weight/Batch (g) |
|---|---|---|---|
| Ajulemic acid | 20.0 | 14.29 | 2.00 |
| Prosolve Easytab | 45.8 | 32.71 | 4.58 |
| Methocel K100M Premium DC | 37.8 | 27.00 | 3.78 |
| Lactose (FastFlo 316) | 21.0 | 15.00 | 2.10 |
| Sodium lauryl sulfate | 14.0 | 10.00 | 1.40 |
| Magnesium stearate | 1.4 | 1.00 | 0.14 |
| Total | 140.0 | 100.00 | 14.00 |

TABLE 3

| Item | mg/Tab | % (w/w) | Weight/Batch (g) |
|---|---|---|---|
| Ajulemic acid | 20.0 | 14.29 | 2.00 |
| Prosolve Easytab | 45.8 | 32.71 | 4.58 |
| Methocel K4M Premium DC | 37.8 | 27.00 | 3.78 |
| Lactose (FastFlo 316) | 21.0 | 15.00 | 2.10 |
| Sodium lauryl sulfate | 14.0 | 10.00 | 1.40 |
| Magnesium stearate | 1.4 | 1.00 | 0.14 |
| Total | 140.0 | 100.00 | 14.00 |

TABLE 4

| Item | mg/Tab | % (w/w) | Weight/Batch (g) |
|---|---|---|---|
| Ajulemic acid | 20.0 | 14.29 | 2.00 |
| Prosolve Easytab | 40.2 | 28.71 | 4.02 |
| Methocel K4M Premium DC | 50.4 | 36.00 | 5.04 |
| Lactose (FastFlo 316) | 21.0 | 15.00 | 2.10 |
| Sodium lauryl sulfate | 7.0 | 5.00 | 0.70 |
| Magnesium stearate | 1.4 | 1.00 | 0.14 |
| Total | 140.0 | 100.00 | 14.00 |

The tablets were made as follows. Ajulemic acid, Prosolv Easytab, Methocel K100M Premium/Methocel K4M Premium, lactose (FastFlo 316), sodium lauryl sulfate, and magnesium stearate were weighed separately. Ajulemic acid, Prosolv Easytab, Methocel K100M Premium/Methocel K4M Premium, lactose (FastFlo 316), and sodium lauryl sulfate were screened through a 30# mesh screen, and magnesium stearate was screened through a 40# mesh screen. Lactose (FastFlo 316) and ajulemic acid (and sodium lauryl sulfate) were charged into a 75 ml HDPE bottle and mixed in a three dimensional pattern for 50 times. Prosolv Easytab and Methocel K100M Premium/Methocel K4M Premium were added to the bottle and mixed in a three dimensional pattern for 100 times. Magnesium stearate was added to the bottle and mixed in a three dimensional pattern for 50 times. The blend was compressed using 7 mm, round standard concave punches on a tablet press at a tablet weight of 140 mg.

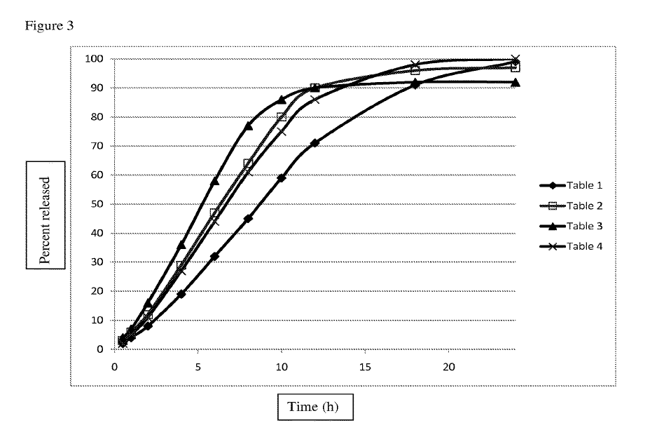

The in-vitro drug release from the extended tablets was studied employing USP Apparatus II (Paddle method) using 900 ml of the dissolution media containing 0.5% w/v of sodium lauryl sulfate. The paddles were operated at 50 rpm, and samples were collected at different time intervals, filtered, and analyzed for ajulemic acid content in the dissolution media by an appropriate analytical method on HPLC. The in-vitro release data and dissolution profiles of ajulemic acid from the tablets are shown in FIG. 3.

### Example 2

### Extended Release Capsule

Extended release pellets were made in a two-stage process: drug layering onto sugar spheres and functional coating with a rate controlling polymer that releases the drug in a controlled manner over a finite period of time. The coated pellets were evaluated for their in-vitro drug release profile using an experimental dissolution method. The drug layering system contained a wetting agent, sodium lauryl sulfate, a binder and plasticizer, hydroxypropyl methylcellulose 5 cps, and polyethylene glycol 20,000, in addition to ajulemic acid. The drug layering system was prepared in water and applied onto the sugar spheres (18/20) in Fluid Bed Coater equipped with a Wurster column. The drug layered pellets were coated with a functional coating suspension of Aquacoat 30% ECD, as a functional polymer to control the drug release from pellets, hydroxypropyl methylcellulose 5 cps (as pore former), and triethyl citrate as plasticizer. Table 5 lists components of exemplary pellets at a 12% weight gain from the coating.

TABLE 5

| Item | Function | % (w/w) | Weight/Batch (g) |
|---|---|---|---|
| Sugar spheres, 18/20 | Inert core | 67.84 | 220.00 |
| Drug Layering | | | |
| Ajulemic acid | Active agent | 11.04 | 36.80 |
| Sodium lauryl sulfate | Wetting agent | 0.25 | 0.80 |
| Hydroxypropyl | Binding agent | 7.40 | 24.0 |
| methylcellulose E5 | | | |
| Polyethylene glycol, | Plasticizer | 1.48 | 4.80 |
| 20000 | | | |
| Purified water | Solvent | N/A | 479.6 |
| Total | | N/A | 285.4 |
| Functional Coating | | | |
| Aquacoat ECD 30% w/w | Rate | 8.33 | 90.00 |
| | controlling | | |
| | polymer | | |
| Triethyl citrate | Plasticizer | 2.00 | 6.48 |
| Hydroxypropyl | Pore former | 1.67 | 5.40 |
| methylcellulose E5 | | | |
| Purified water | Solvent | N/A | 157.2 |
| Total | | 100.00 | 324.3 |

Sugar spheres (18/20), ajulemic acid, sodium lauryl sulfate, hydroxypropyl methylcellulose E5 (HPMC E5), polyethylene glycol 20,000 (PEG 20,000), and purified water were weighed separately. Sodium lauryl sulfate (SLS) was added to purified water and mixed with an impeller blade until a clear solution formed. Ajulemic acid was dispersed in SLS solution using a SiIverson homogenizer until a uniform suspension formed. HPMC E5 and PEG 20,000 were added to purified water and mixed with an impeller blade until a clear solution formed. The drug suspension was added to the HPMC solution and mixed with an impeller blade for 10 minutes until a uniform suspension formed. The 18/20 mesh sugar spheres were charged into the fluid bed bowl and sprayed using a Wurster column assembly to a determined weight gain, and the layered spheres were dried. The final dried spheres were screened using a screen stack of 14 and 20 mesh screens and collected.

Aquacoat ECD 30%, triethyl citrate (TEC), HPMC E5, and purified water were weighed separately, HPMC E5 was added to purified water and mixed with an impeller blade until a clear solution formed. Aquacoat ECD 30% was added to the mixing vessel and mixed with a propeller blade for 5 minutes. TEC was added to the Aquacoat HPMC dispersion, and mixing continued for an additional 30 minutes. Drug layered pellets were placed into the fluid bed, and the functional coating suspension was sprayed to a pre-determined weight gain. The coated pellets were dried for about 15 minutes at a bed temperature of 30-36° C. The coated pellets were cured for 2 hrs in an oven at 60° C. The final dried pellets were screened using a screen stack of 14 and 20 mesh screens and collected.

The in-vitro drug release from the pellets is shown in Table 6.

TABLE 6

| | Cumulative Percent Drug Released | |
|---|---|---|
| Time (Hrs) | 9% Weight Gain Pellets | 12% Weight Gain Pellets |
| 0.5 | 14 | 1 |
| 1 | 26 | 5 |
| 2 | 44 | 11 |
| 4 | 67 | 24 |
| 6 | 82 | 36 |
| 8 | 93 | 47 |
| 10 | 100 | 57 |
| 12 | 100 | 65 |
| 18 | 100 | 86 |
| 24 | 100 | 100 |

The dissolution data depicted in Table 6 show an extended release of drug over 24 hrs. Further the analytical data revealed that the drug release has decreased with increasing the functional coating weight gain from 9% to 12% w/w.

A number of embodiments of the invention have been described. Nevertheless, it will be understood that various modifications may be made without departing from the spirit and scope of the invention. Accordingly, other embodiments of a sustained release formulation and method of administration are within the scope of the following claim.

Other embodiments are in the claims.

What is claimed is:

1. A sustained release pharmaceutical composition comprising a therapeutically or prophylactically effective amount of a compound of formula I: 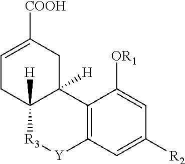
wherein R₁ is hydrogen, COCH₃ or COCH₂CH₃; R₂ is a branched C₅-C₁₂ alkyl group that may optionally have a terminal aromatic ring, or optionally a branched OCHCH₃(CH₂)ₘCH₃ alkyl group that may have a terminal aromatic ring, wherein m is 0 to 7; R₃ is hydrogen, a C₁₋₈ alkyl group or a C₁₋₈ alkanol group; and Y is nil or a bridging group of NH or oxygen, provided that where Y is oxygen and R₂ is a branched C₅₋₁₂ alkyl, R₃ is not CHCH₃, or a pharmaceutically acceptable salt, ester, or solvate thereof and a pharmaceutically acceptable carrier.

2. The pharmaceutical composition of claim 1, wherein administration of the composition results in a substantial reduction in an adverse event or risk thereof.

3. The pharmaceutical composition of claim 2, wherein the adverse event is one or more of dizziness, dry mouth, disorientation, euphoria, headache, nausea, pallor, somnolence, vomiting, tremor, abnormal feeling, tachycardia, fatigue, feeling drunk, paraesthesia, muscle spasms, muscle tightness, disturbance in attention, déjà vu, altered mood, anorexia, and cardiovascular events such as orthostatic hypotension, or QTc prolongation.

4. The pharmaceutical composition of any preceding claim, wherein the composition provides a therapeutically effective or prophylactically effective amount when administered once daily.

5. The pharmaceutical composition of any preceding claim, wherein the composition provides a therapeutically effective or prophylactically effective amount when administered twice daily.

6. The pharmaceutical composition of any preceding claim, wherein the composition provides a therapeutically effective or prophylactically effective amount when administered three times daily.

7. The pharmaceutical composition of any preceding claim, wherein the therapeutically or prophylactically effective amount is released over at least 8 hours.

8. The pharmaceutical composition of any preceding claim, wherein the therapeutically or prophylactically effective amount is released over at least 12 hours.

9. The pharmaceutical composition of any preceding claim, wherein the therapeutically or prophylactically effective amount is 5 to 240 mg.

10. The pharmaceutical composition of any preceding claim, wherein the therapeutically or prophylactically effective amount is 5 to 180 mg.

11. The pharmaceutical composition of any preceding claim, wherein the therapeutically or prophylactically effective amount is 5 to 120 mg.

12. The pharmaceutical composition of any preceding claim, wherein the therapeutically or prophylactically effective amount is from 20 to 120 mg.

13. The pharmaceutical composition of any preceding claim, wherein the maximum exposure to the compound of formula I, based on AUC₀₋₂₄ₕᵣ, is less than 36,000 ng-hr/ml.

14. The pharmaceutical composition of any preceding claim, wherein the maximum exposure to the compound of formula I, based on AUC₀₋₂₄ₕᵣ, is less than 25,000 ng-hr/ml.

15. The pharmaceutical composition of any preceding claim, wherein the minimum exposure to the compound of formula I, based on AUC₀₋₂₄ₕᵣ, is 5,000 ng-hr/ml.

16. The pharmaceutical composition of any preceding claim, wherein the maximum exposure to the compound of formula I, based on Cₘₐₓ, is less than 2500 ng/ml.

17. The pharmaceutical composition of any preceding claim, wherein the maximum exposure to the compound of formula I, based on Cₘᵢₙ, is less than 1200 ng/ml.

18. The pharmaceutical composition of any preceding claim, wherein the therapeutic or prophylactic effect is maintained by administration of up to three doses per day of the pharmaceutical composition.

19. The pharmaceutical composition of any preceding claim, wherein the minimum exposure to the compound of formula I, based on Cₘᵢₙ, is 100 to 500 ng/ml.

20. The pharmaceutical composition of any preceding claim, wherein the therapeutically or prophylactically effective amount is 5 to 60 mg, the maximum exposure to the compound of formula I based on AUC₀₋₂₄ is 5,000 to 30,000 ng-hr/ml, based on Cₘₐₓ is less than 1200 ng/ml, and based on Cₘᵢₙ is 100 to 500 ng/ml.

21. The pharmaceutical composition of any preceding claim, wherein Tₘₐₓ is less than 4 hours.

22. The pharmaceutical composition of any preceding claim, wherein the compound of formula I is ajulemic acid.

23. A method of delivering a compound of formula I, said method comprising administering the pharmaceutical composition of any of claims 1 to 22 to a subject in need thereof.

24. A method of delivering a compound of formula I, said method comprising administering a therapeutically or prophylactically effective amount of a compound of formula I: 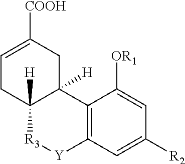
wherein R₁ is hydrogen, COCH₃ or COCH₂CH₃; R₂ is a branched C₅-C₁₂ alkyl group that may optionally have a terminal aromatic ring, or optionally a branched OCHCH₃(CH₂)ₘCH₃ alkyl group that may have a terminal aromatic ring, wherein m is 0 to 7; R₃ is hydrogen, a C₁₋₈ alkyl group or a C₁₋₈ alkanol group; and Y is nil or a bridging group of NH or oxygen, provided that where Y is oxygen and R₂ is a branched C₅₋₁₂ alkyl, R₃ is not CHCH₃, or a pharmaceutically acceptable salt, ester, or solvate thereof, wherein
(a) administration of the compound of formula I results in a substantial reduction in an adverse event or risk thereof; and/or
(b) the compound of formula I is administered in a sustained release formulation.

25. The method of claim 24, wherein
(i) the maximum exposure to the compound of formula I, based on AUC₀₋₂₄ₕᵣ, is less than 36,000 ng-hr/ml;
(ii) the minimum exposure to the compound of formula I, based on AUC₀₋₂₄ₕᵣ, is 5000 ng-hr/ml;
(iii) the maximum exposure to the compound of formula I, based on Cₘₐₓ, is less than 2500 ng/ml;
(iv) the maximum exposure to the compound of formula I, based on Cₘᵢₙ, is less than 1200 ng/ml; and/or
(v) the minimum exposure to the compound of formula I, based on Cₘᵢₙ, is 100 to 500 ng/ml.

26. The method of claim 24, wherein the therapeutically or prophylactically effective amount is 5 to 60 mg, the maximum exposure to the compound of formula I based on AUC₀₋₂₄ is 5,000 to 30,000 ng-hr/ml, based on Cₘₐₓ is less than 1200 ng/ml, and based on Cₘᵢₙ is 100 to 500 ng/ml.

27. The method of any of claims 24-26, wherein Tₘₐₓ is less than 4 hours.

28. The method of any of claims 24-27, wherein the adverse event is one or more of dizziness, dry mouth, disorientation, euphoria, headache, nausea, pallor, somnolence, vomiting, tremor, abnormal feeling, tachycardia, fatigue, feeling drunk, paraesthesia, muscle spasms, muscle tightness, disturbance in attention, déjà vu, altered mood, anorexia, and cardiovascular events such as orthostatic hypotension, or QTc prolongation.

29. The method of any of claims 23-28, wherein the administering is for the treatment or prevention of a CB1/CB2 associated disease.

30. The method of any of claims 23-29, wherein the administering is for the treatment or prevention of pain or inflammation.

31. The method of any of claims 23-30, wherein the subject is diagnosed with pain or inflammation.

32. The method of any of claims 23-31, wherein the administering is for the treatment or prevention of a condition selected from the group consisting of osteoarthritis, systemic lupus erythematosus, post herpetic neuralgia, neuropathic pain, diabetic neuropathy, lower back pain, multiple sclerosis, cystitis, rheumatoid arthritis, Crohn's Disease, inflammatory bowel disease (IBD), psoriasis, psoriatic arthritis, ankylosing spondylitis, vasculitis, myositis, Muckle-Wells Syndrome, and Familial Mediterranean Fever (FMF).

33. The method of any of claims 23-32, wherein the administering is for the treatment or prevention of a condition selected from the group consisting of neck pain, shoulder pain, temporomandibular joint pain, chronic pelvic pain, complex regional pain syndrome, musculoskeletal pain, osteoarthritis pain, rheumatoid arthritis pain, fibromyalgia pain, cancer pain, chemotherapy-induced neuropathy pain, neuropathy pain secondary to tumor-infiltration, trigeminal neuralgia pain, postherpetic neuralgia pain, phantom limb pain, HIV sensory neuropathy pain, HIV myelopathy pain, peripheral diabetic neuropathy pain, gout pain, pain with irritable bowel syndrome, bladder pain, central post-stroke pain, multiple sclerosis pain, spinal cord injury pain, sciatic nerve pain, post ischemic myelopathy pain, orthopedic pain, pain from cardiovascular disease, breast pain, psoriasis, eczema, dermatitis, burn, peripheral neuropathic and central neuropathic pain, chronic pain, crush injury and trauma induced pain, cerebrovascular and vascular pain, sickle cell disease pain, orofacial and facial pain including dental, surgical incision related pain, visceral pain, soft tissue inflammatory pain, reflex sympathetic dystrophy, and pain resulting from kidney stones or urinary tract infection.

34. The method of any of claims 23-33, wherein the compound of formula I is administered to the subject over a period of time of at least 8, 10, 14, 21, 28, 60, 90, 120, or 365 days either continuously, intermittently or with some interruptions.

35. The method of any of claims 23-34, wherein the compound of formula I is administered once daily.

36. The method of any of claims 23-35, wherein the compound of formula I is administered twice daily.

37. The method of any of claims 23-36, wherein the compound of formula I is administered thrice daily.

38. The method of any of claims 23-37, wherein the compound of formula I is administered at least once daily for at least 7, 14, or 30 days.

39. The method of any of claims 23-38, wherein the amount of the compound of formula I administered is from 5-240 mg/day, 5-180 mg/day, 5-120 mg/day, or 20-120 mg/day.

40. The method of any of claims 23-39, wherein the compound of formula I is ajulemic acid.

41. A compound of formula I: 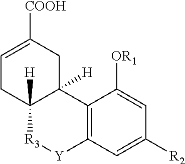
wherein R₁ is hydrogen, COCH₃ or COCH₂CH₃; R₂ is a branched C₅-C₁₂ alkyl group that may optionally have a terminal aromatic ring, or optionally a branched OCHCH₃(CH₂)ₘCH₃ alkyl group that may have a terminal aromatic ring, wherein m is 0 to 7; R₃ is hydrogen, a C₁₋₈ alkyl group or a C₁₋₈ alkanol group; and Y is nil or a bridging group of NH or oxygen, provided that where Y is oxygen and R₂ is a branched C₅₋₁₂ alkyl, R₃ is not CHCH₃, or a pharmaceutically acceptable salt, ester, or solvate thereof
for use in a method of treating or preventing a CB1/CB2 associated disease, wherein
(a) administration of the compound of formula I results in a substantial reduction in an adverse event or risk thereof; and/or
(b) the compound of formula I is administered in a sustained release formulation.

42. The compound for use of claim 41, wherein
(i) the maximum exposure to the compound of formula I, based on AUC₀₋₂₄ₕᵣ, is less than 36,000 ng-hr/ml;
(ii) the minimum exposure to the compound of formula I, based on AUC₀₋₂₄ₕᵣ, is 5000 ng-hr/ml;
(iii) the maximum exposure to the compound of formula I, based on Cₘₐₓ, is less than 2500 ng/ml;
(iv) the maximum exposure to the compound of formula I, based on Cₘᵢₙ, is less than 1200 ng/ml; and/or
(v) the minimum exposure to the compound of formula I, based on Cₘᵢₙ, is 100 to 500 ng/ml.

43. The compound for use of claim 41 or 42, wherein the therapeutically or prophylactically effective amount is 5 to 60 mg, the maximum exposure to the compound of formula I based on AUC₀₋₂₄ is 5000 to 30,000 ng-hr/ml, based on Cₘₐₓ is less than 1200 ng/ml, and based on Cₘᵢₙ is 100 to 500 ng/ml.

44. The compound for use of any of claims 41-43, wherein Tₘₐₓ is less than 4 hours.

45. The compound for use of any of claims 41-44, wherein the subject is diagnosed with pain or inflammation.

46. The compound for use of any of claims 41-45, wherein the CB1/CB2 associated disease is pain or inflammation.

47. The compound for use of any of claims 41-46, wherein the CB1/CB2 associated disease is selected from the group consisting of osteoarthritis, systemic lupus erythematosus, post herpetic neuralgia, neuropathic pain, diabetic neuropathy, lower back pain, multiple sclerosis, cystitis, rheumatoid arthritis, Crohn's Disease, inflammatory bowel disease (IBD), psoriasis, psoriatic arthritis, ankylosing spondylitis, vasculitis, myositis, Muckle-Wells Syndrome, and Familial Mediterranean Fever (FMF).

48. The compound for use of any of claims 41-47, wherein CB1/CB2 associated disease is selected from the group consisting of neck pain, shoulder pain, temporomandibular joint pain, chronic pelvic pain, complex regional pain syndrome, musculoskeletal pain, osteoarthritis pain, rheumatoid arthritis pain, fibromyalgia pain, cancer pain, chemotherapy-induced neuropathy pain, neuropathy pain secondary to tumor-infiltration, trigeminal neuralgia pain, postherpetic neuralgia pain, phantom limb pain, HIV sensory neuropathy pain, HIV myelopathy pain, peripheral diabetic neuropathy pain, gout pain, pain with irritable bowel syndrome, bladder pain, central post-stroke pain, multiple sclerosis pain, spinal cord injury pain, sciatic nerve pain, post ischemic myelopathy pain, orthopedic pain, pain from cardiovascular disease, breast pain, psoriasis, eczema, dermatitis, burn, peripheral neuropathic and central neuropathic pain, chronic pain, crush injury and trauma induced pain, cerebrovascular and vascular pain, sickle cell disease pain, orofacial and facial pain including dental, surgical incision related pain, visceral pain, soft tissue inflammatory pain, reflex sympathetic dystrophy, and pain resulting from kidney stones or urinary tract infection.

49. The compound for use of any of claims 41-48, wherein the compound of formula I is administered to the subject over a period of time of at least 8, 10, 14, 21, 28, 60, 90, 120, or 365 days either continuously, intermittently or with some interruptions.

50. The compound for use of any of claims 41-49 wherein the compound of formula I is administered once daily.

51. The compound for use of any of claims 41-50 wherein the compound of formula I is administered twice daily.

52. The compound for use of any of claims 41-51, wherein the compound of formula I is administered thrice daily.

53. The compound for use of any of claims 41-52, wherein the compound of formula I is administered at least once daily for at least 7, 14, or 30 days.

54. The compound for use of any of claims 41-53, wherein the amount of ajulemic acid administered is from 5-240 mg/day, 5-180 mg/day, 5-120 mg/day, or 20-120 mg/day.

55. The compound for use of any of claims 41-54, wherein the adverse event is one or more of dizziness, dry mouth, disorientation, euphoria, headache, nausea, pallor, somnolence, vomiting, tremor, abnormal feeling, tachycardia, fatigue, feeling drunk, paraesthesia, muscle spasms, muscle tightness, disturbance in attention, déjà vu, altered mood, anorexia, and cardiovascular events such as orthostatic hypotension, or QTc prolongation.

56. The compound for use of any of claims 41-55, wherein the compound of formula I is ajulemic acid.
